(19) United States
(12) Patent Application Publication
(10) Pub. No.: US 2023/0120737 A1
(43) Pub. Date: Apr. 20, 2023

(54) SYSTEMS AND METHODS FOR PERFORMING A MINIMALLY INVASIVE VASECTOMY PROCEDURE

(71) Applicant: Robert F. Rioux, Ashland, MA (US)

(72) Inventor: Robert F. Rioux, Ashland, MA (US)

(21) Appl. No.: 17/966,692

(22) Filed: Oct. 14, 2022

Related U.S. Application Data
(60) Provisional application No. 63/255,757, filed on Oct. 14, 2021.

(51) Int. Cl. A61B 18/14; A61B 8/08; A61B 8/00; A61F 6/20

(52) U.S. Cl. CPC A61B 18/1477; A61B 8/0841; A61B 8/4455; A61F 6/20; A61B 2018/126

(57) ABSTRACT

The invention provides systems and methods for performing a minimally invasive vasectomy procedure by supplying targeted RF energy to vas deferens via a pair of probes positioned within the vas deferens under ultrasound image guidance.

### CROSS-REFERENCE TO RELATED APPLICATIONS

This application claims priority to, and the benefit of, U.S. Provisional Application No. 63/255,757, filed Oct. 14, 2021, the content of which is incorporated by reference herein in its entirety.

### TECHNICAL FIELD

The invention generally relates to male sterilization devices and techniques, and, more particularly, to a system providing a means for performing a minimally invasive vasectomy procedure by supplying targeted radiofrequency (RF) energy to vas deferens via a pair of probes positioned within the vas deferens under ultrasound image guidance.

### BACKGROUND

Male sterilization is generally accomplished by vasectomy, in which the ducts that carry sperm out of the testes (i.e., the vas deferens) are surgically interrupted by ligation and/or by cauterization, thereby stopping the flow of sperm from the testicle to the prostate gland. As a result, the female egg cannot be fertilized after intercourse. This procedure generally requires a surgical opening of the scrotum. Ideally, a vasectomy is an outpatient procedure that is desirably completed with mild discomfort for the patient. The patient should then be capable of resuming his normal activities within a reasonable time frame. The majority of cases have this degree of successful results and limited aftereffects.

In a significant number of instances, however, prolonged exploration and manipulation accompanied by excessive discomfort both intraoperatively and postoperatively can make the results less than desirable. Complications can arise, at least in part, because scrotal tissue is highly elastic. Whereas a small amount of bleeding is quickly stopped by the tension that develops in non-elastic tissue, elastic tissue offers little pressure to slow the loss of blood and fluid. Thus, even the slightest amount of persistent bleeding can cause tremendously large hematomas. As a result, and in addition to causing discomfort, the healing process is slowed because of the prolonged time required to reabsorb these fluids and cells, increasing the opportunity for bacterial colonization.

In addition, another concern for the surgeon is the elusiveness of the vas deferens. In current vasectomy procedures, the vas deferens are not seen until the later stages of the procedure, and must be identified by palpation. Once identified and delivered into the operative field, the vas deferens must be held in place by some means of fixation. Even a momentary release of the vas allows it to immediately return to within the spermatic cord, from which it must again be extricated. Furthermore, the injection of a local anesthetic into the scrotal skin and the area surrounding the vas makes palpation of the structure difficult. Loss of fixation of the vas can result in the need for increased dissection, and, as a result, such manipulation can cause increased bleeding and swelling.

### SUMMARY

The present invention recognizes the disadvantages of current vasectomy procedures and related surgical devices, specifically the drawbacks associated with incision- or puncture-based procedures for accessing the vas deferens resulting in discomfort and pain, as well as lingering effects, such as bacterial infection and increased time of recovery, as well as the difficulties in locating and handling the vas deferens, leading to increased time of procedure and potential inaccuracy when attempting to ligate and/or by cauterize the vas deferens.

In particular, the present invention is directed to a system providing a means for performing a minimally invasive vasectomy procedure by supplying targeted RF energy to vas deferens via a pair of probes that are positioned within the vas deferens under ultrasound image guidance.

The system includes an electrosurgical device including a guide bracket member and a needle carriage operably coupled to the guide bracket member and comprising at least a pair of needle probes (forming a bipolar electrode) for delivering RF energy to a targeted tissue, specifically the vas deferens. The guide bracket member is configured to be releasably coupled to an imaging modality probe, such as, for example, a handheld ultrasound transducer probe. More specifically, the guide bracket member can be fitted over a distal operating end of the transducer probe without impeding the performance of the probe (i.e., without interrupting the transmission of sound waves to and from the transducer probe that are used for creating an ultrasound image of the tissue undergoing examination). The guide bracket member is configured to receive and releasably retain the needle carriage thereto at an acute angle relative to the ultrasound transducer probe distal end. Such an orientation allows for the needle probes, particularly the penetrating distal ends thereof, to move in a direction aimed toward a target site generally centered with the distal operating end of the ultrasound transducer probe. Accordingly, when performing the procedure, the user can better target a center of the vas deferens when moving the needle probes to a fully deployed configuration, such that the tips of the probes can extend into the vas deferens and be positioned within the vas lumen for subsequent delivery of RF energy thereto.

In response to input from an operator of the device (i.e., surgeon or other medical professional carrying out the procedure), the needle carriage is configured to move relative to the guide bracket member to allow for advancement and retraction of the needle carriage, and, in turn, the needle probes, relative to a target site, specifically the vas deferens, during an ultrasound imaging procedure used in visually locating the vas deferens. In particular, the needle carriage is configured to move relative to the guide bracket member via a rack and pinion assembly, which provides movement of the needle carriage between a fully retracted configuration (in which the needle probes are fully withdrawn and do not extend past the operating distal end of the transducer probe) and a fully deployed configuration (in which the needle probes are fully extended past the operating distal end of the transducer probe). The rack and pinion assembly includes, for example, a set of gears (i.e., pinion gears of the like) for receiving input from a user-operated input, which may be in the form of a knob, and converting the input (i.e., rotation of the knob) to linear motion of a rack member provided on the needle carriage.

The electrosurgical device may be configured to allow for different degrees of adjustment of the needle carriage, such as coarse movement/adjustment (i.e., faster and less precise linear movement of the needle probes between retracted and deployed configurations) and fine movement/adjustment (i.e., slower and more precise linear movement of the needle probes between retracted and deployed configurations). Accordingly, the needle probes can be advanced into the vas deferens with precise control and accuracy. For example, the electrosurgical device may further include a drive lever associated with the rack and pinion assembly that is movable between a disengaged and an engaged state. For example, the drive lever may be operably coupled with the pinion gear, and, when moved to a disengaged state, the pinion gear and rack member are disengaged and the user can simply push or pull the needle carriage to the desired position in a coarse movement/adjustment. When the drive lever is moved to an engaged state, the pinion gear and rack member are engaged such that a user can rotate the knob (associated with the pinion gear) to cause the needle carriage, and specifically the needle probes, to move to the desired position in a fine movement/adjustment.

The electrosurgical device, specifically the pair of needle probes of the needle carriage, are electrically coupled to an electrosurgical generator for providing bipolar low power (i.e., between 1 and 20 watts or more) RF energy delivery. The system of the present invention further includes a controller communicatively coupled to the electrosurgical device and the electrosurgical generator. The controller may be configured to initiate, terminate, and/or adjust delivery of RF energy from the pair of needle probes provided by the electrosurgical device. In some embodiments, the controller may include a temperature control module, for example, that is configured to receive temperature readings from a temperature sensor provided on the electrosurgical device and positioned adjacent to target site. In particular, the temperature control module may be configured to continuously monitor the temperature of a patient's skin at or near the target site during RF energy delivery to the vas deferens from the pair of needle probes. Upon reaching a desired maximum temperature (i.e., a predetermined temperature at which there is a high correlation with successful coagulation of the vas deferens), the temperature control module is configured to automatically terminate delivery of RF energy so as to prevent unnecessary collateral damage to surrounding tissue.

Furthermore, the electrosurgical device may be single use, while the generator and controller are reusable. As such, the system may include a means for authenticating a given electrosurgical device to determine whether the electrosurgical device is suitable and/or authorized to operate with the controller and/or generator. In particular, the temperature control module may include an RFID reader, for example, for reading data embedded in an RFID tag associated with the electrosurgical device upon attachment of the electrosurgical device to the controller. The data from the RFID tag may be analyzed by the controller and a determination can then be made as to whether the electrosurgical device is authentic and whether it has been previously used. In the event that the electrosurgical device is determined to be authentic and not previously used, the control system allows for transmission of power to the electrosurgical device and thus a procedure can be performed using the needle probes. In the event that the electrosurgical device is determined to not be authentic or that it already has been used, the controller prevents transmission of energy to the electrosurgical device.

Accordingly, the electrosurgical device described herein is used to perform a vasectomy under ultrasound image guidance in a highly accurate and minimally invasive manner. In particular, because the electrosurgical device is directly coupled to an ultrasound transducer probe, a surgeon can perform a vasectomy in conjunction with an ultrasound imaging procedure of the target site, which allows for the surgeon to view the target site (i.e., the spermatic cord) in real-time without having to create unnecessary incisions or punctures and further locate and target the vas deferens when piercing the skin and advancing the needle probes directly into the vas lumen in a precise manner (via the fine tune advancement feature). Furthermore, the present invention provides for an automatic control over the delivery of RF energy based on continuous temperature readings at the target site to ensure that the risk of unnecessary collateral damage is minimized and that the procedure is carried out to completion. As such, the systems and methods of the present invention greatly reduce the chance of infection, bleeding, and scrotal pain associated with current techniques and devices, which may lead to greater acceptance of vasectomy by men, reducing the morbidity, mortality, and cost associated with tubal ligation.

### BRIEF DESCRIPTION OF THE DRAWINGS

FIGS. 1A and 1B are diagrammatic illustrations of a system for performing a minimally invasive vasectomy procedure according to embodiments of the present disclosure.

FIG. 2 shows an exemplary electrosurgical device according to one embodiment of the present disclosure operably coupled to a conventional ultrasound transducer probe and an exemplary controller for controller delivery of energy to the electrosurgical device.

FIGS. 3A and 3B illustrate one embodiment of a guide bracket member of the electrosurgical device transitioning from a disassembled state (FIG. 3A) to an assembled state (FIG. 3B) and fitted over an ultrasound transducer probe.

FIG. 4 is an exploded perspective view of an electrosurgical device consistent with the present disclosure, specifically depicting various components of the guide bracket member.

FIG. 5 is a perspective view of one embodiment of a needle carriage consistent with the present disclosure.

FIG. 6A is a perspective view and FIG. 6B is a perspective view, partly in section, of an exemplary embodiment of a guide bracket member and needle carriage illustrating the manner in which the needle carriage is releasably and movably coupled to the guide bracket member.

FIGS. 7A and 7B are perspective and side sectional views of portions of the guide bracket member and the needle carriage illustrating a drive lever in a disengaged position.

FIG. 7C shows an ultrasound transducer probe equipped with an exemplary electrosurgical device consistent with the present disclosure and having a drive lever in the disengaged position to thereby allow for course movement/adjustment of the needle carriage, including the pair of needle probes, relative to the target site.

FIGS. 8A and 8B are perspective and side sectional views of portions of the guide bracket member and the needle carriage illustrating a drive lever in a engaged position.

FIG. 8C shows an ultrasound transducer probe equipped with an exemplary electrosurgical device consistent with the present disclosure and having a drive lever in the engaged position to thereby allow for fine tune movement/adjustment of the needle carriage, including the pair of needle probes, relative to the target site.

FIGS. 9A-9C are illustrated representations of steps for performing vasectomy procedure with systems consistent with the present disclosure. FIG. 9A illustrates initial grasping of the vas deferens by the physician and brought up to a position just below the surface of scrotum, in which physician may be standing on one side of the patient while the patient is lying on an exam table, and the electrosurgical device fitted over an ultrasound transducer probe is positioned relative to the vas deferens. FIG. 9B illustrates locating and penetrating the vas deferens with the needle probes under ultrasound image guidance, in which a physician can view, in real-time, an ultrasound image, as shown in FIG. 9C, of the vas deferens and advancement of the needle probes into the vas lumen.

### DETAILED DESCRIPTION

By way of overview, the present invention is directed to systems and methods performing a minimally invasive vasectomy procedure by supplying targeted RF energy to vas deferens via a pair of probes positioned within the vas deferens under ultrasound image guidance.

It should be noted that, while the systems and methods described herein are directed to sterilizing a male patient via a vasectomy procedures, the systems and methods can be used to for supplying energy for cauterizing/coagulating any tubular vessel of a patient. As such, the systems and methods of the present disclosure are not limited to vasectomy procedures and, rather, may be used for any procedure requiring occlusion of a vessel (by cautery and/or coagulating means) and in a minimally invasive manner.

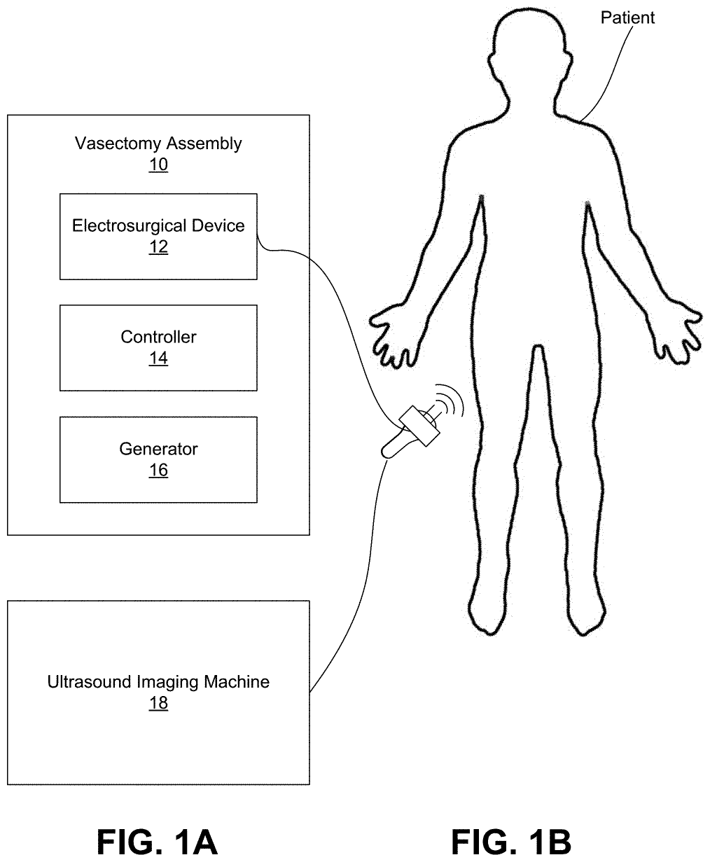

FIGS. 1A and 1B are diagrammatic illustrations of a system for performing a minimally invasive vasectomy procedure on a patient according to embodiments of the present disclosure. The system includes a vasectomy assembly 10, including an electrosurgical device 12, a controller 14, and a generator 16 to which the electrosurgical device 12 is to be operably connected for receiving energy therefrom (wherein such energy supply can be controlled via the controller 14).

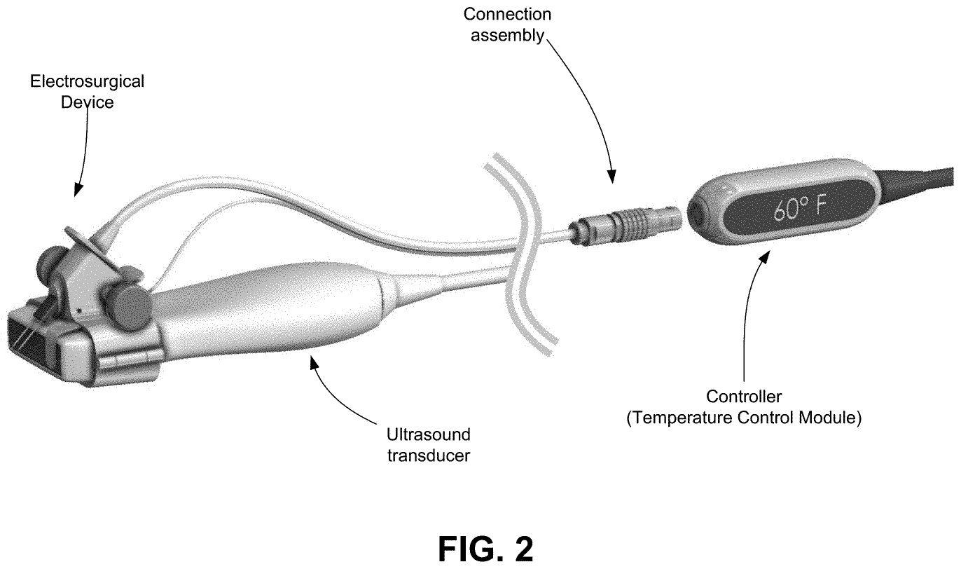

The system of the present invention further includes an ultrasound imaging machine 18 to which the electrosurgical surgical device 12 is to be coupled. In particular, the electrosurgical device 12 is configured to be fitted to a conventional handheld ultrasound transducer probe such that, during a procedure, the operator (i.e., the surgeon or other medical professional), is simultaneously performing an ultrasound imaging procedure (i.e., utilizing the transducer probe to obtain an ultrasound image of the target site) and a vasectomy procedure (i.e., utilizing the electrosurgical device to target and deliver energy directly to the vas deferens under the ultrasound image guidance). For example, FIG. 2 shows an exemplary electrosurgical device according to one embodiment of the present disclosure operably coupled to a conventional ultrasound transducer probe and an exemplary controller for controller delivery of energy to the electrosurgical device.

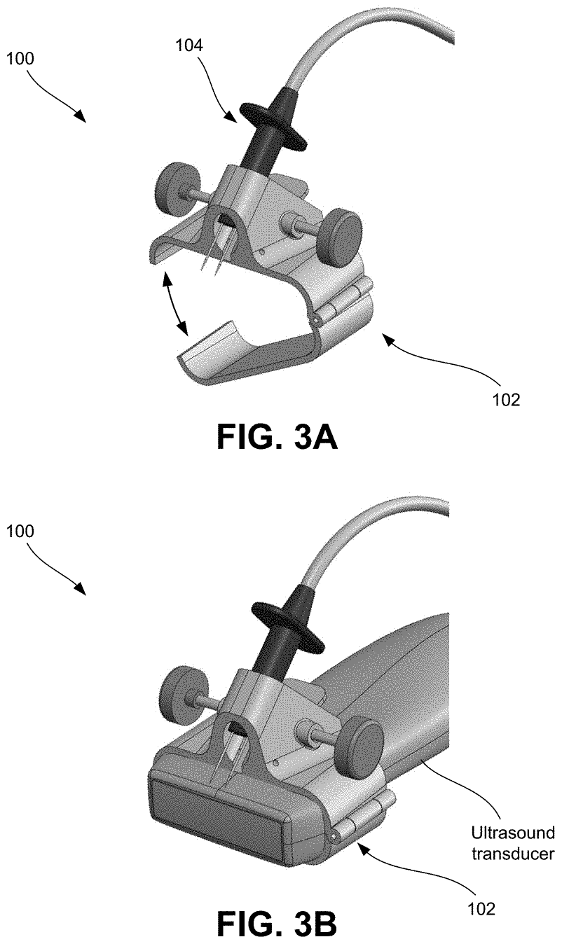

As shown in FIGS. 3A and 3B, the electrosurgical device 100 generally includes a guide bracket member 102 and a needle carriage 104 operably coupled to the guide bracket member 102. The needle carriage 104 includes at least a pair of needle probes (forming a bipolar electrode) for delivering RF energy to targeted tissue, specifically the vas deferens when performing a vasectomy. The guide bracket member 102 is configured to be releasably coupled to an imaging modality probe, such as, for example, a handheld ultrasound transducer probe. More specifically, the guide bracket member 102 can be fitted over a distal operating end of the transducer probe without impeding the performance of the probe (i.e., without interrupting the transmission of sound waves to and from the transducer probe that are used for creating an ultrasound image of the tissue undergoing examination).

For example, FIGS. 3A and 3B illustrate one embodiment of a guide bracket member 102 of the electrosurgical device transitioning from a disassembled state (FIG. 3A) to an assembled state (FIG. 3B) and fitted over an ultrasound transducer probe. As shown, the guide bracket member 102 may include an aperture shaped and/or sized to fit snugly over the operating distal end of the transducer probe. In some embodiments, the guide bracket member 102 may be composed of at least two pieces that are hingedly attached to one another and cooperatively form the aperture (for receiving the transducer probe) when coupled to one another. For example, as shown in FIG. 3A, a portion of the guide bracket member is in a disassembled state in which a hinged portion is unconnected to the remainder of the body of the guide bracket member. Accordingly, a user need only place the hinged portion around the transducer probe and lock it into place (i.e., via any known coupling mechanism, such as a snap-fit coupling mechanism or the like). It should be noted that, in some embodiments, the aperture may be preformed and the guide bracket member body is of a monolith construction, such that the guide bracket member is coupled directly to the transducer probe via a press-fit coupling (sliding of the guide bracket member over the transducer probe for a snug fit). It should further be noted that, in some embodiments, the guide bracket member my further include an insert (positioned within the aperture) comprising a deformable material that accommodates a variety of different shaped and/or sized ultrasound transducer probes, while still providing a secure engagement between the guide bracket member and the probe.

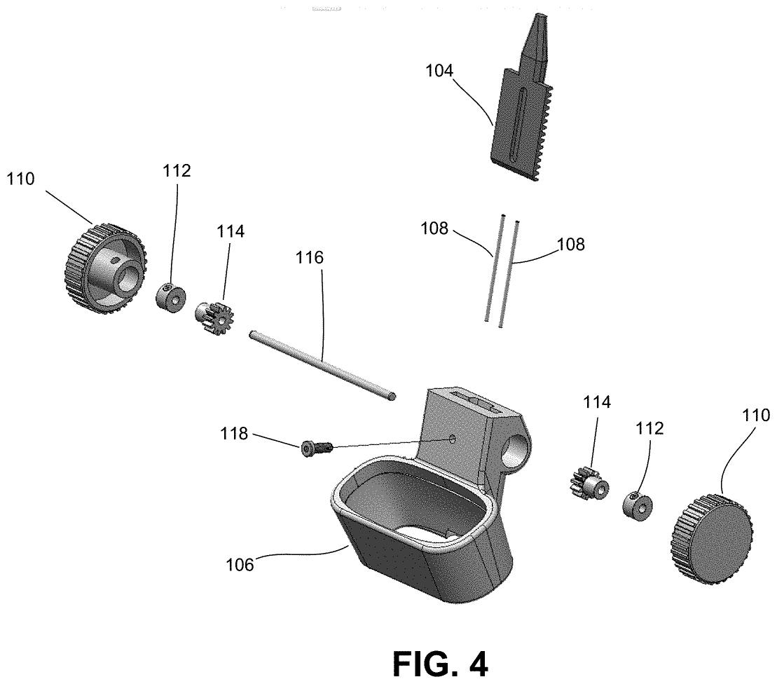

FIG. 4 is an exploded perspective view of an electrosurgical device 100 consistent with the present disclosure, specifically depicting various components of the guide bracket member 102 and the needle carriage 104. For example, as shown, the guide bracket member 102 may include a main body 106 including a first aperture through which the transducer probe can be received and a second aperture for receiving at least a portion of the needle carriage 104 allowing for linear movement of the needle carriage 104 therethrough, as will be discussed in greater detail herein. The main body 106 may be constructed of any medical grade material. However, it should be noted that, in some embodiments, the guide bracket member and the needle carriage are intended to be single use and, as such, the materials from which they are made need not be intended for reuse.

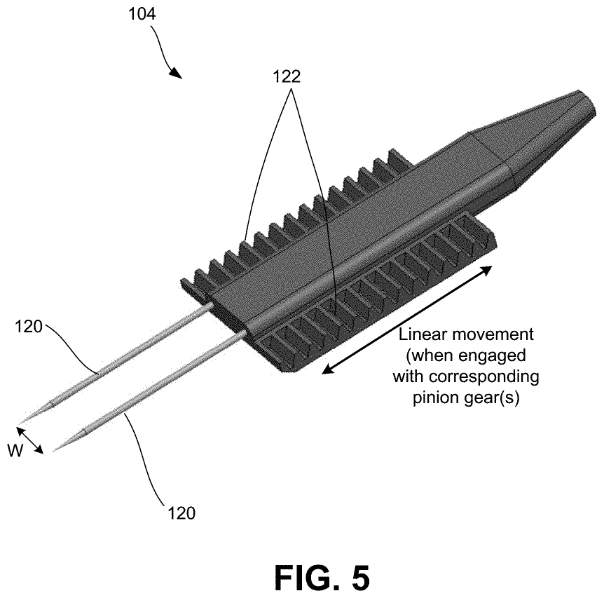

As previously described herein, needle carriage 104 includes a pair of needle probes. For example, FIG. 5 provides a perspective view of one embodiment of a needle carriage 104 consistent with the present disclosure, in which a pair of needle probes 120 are shown attached thereto. Depending on the application, the needle probes 120 may be spaced a specific distance apart (shown as width W). For example, in a preferred embodiment, the width between the needle probes 120 may be approximately 5 mm. However, the width W may be between 1 mm and 10 mm. Furthermore, the needle probes 120 may have thickness of between 10 gauge and 50 gauge. For example, in a preferred embodiment, the needle probes 120 have a 25 gauge thickness.

Referring to FIG. 4, stainless steel dowel pins 108 used in place of the needle probes and may similarly deliver RF energy at the desired level for a similar effect. However, by providing needle probes (specifically having a 25 gauge thickness and spaced approximately 5 mm apart, as well as delivering RF energy at 6 W (or 10 W), the inventors found that such needle probe specifications provided optimal coagulation at the target site, in that the needle probes are were able to penetrate tissue (i.e., skin and vas deferens) with little resistance and deliver sufficient energy to cause adequate and complete coagulation as a result of the bipolar probe configuration.

In response to input from an operator of the device (i.e., surgeon or other medical professional carrying out the procedure), the needle carriage 104 is configured to move relative to the guide bracket member 102 to allow for advancement and retraction of the needle carriage 104, and, in turn, the needle probes 120, relative to a target site, specifically the vas deferens, during an ultrasound imaging procedure used in visually locating the vas deferens.

In particular, the needle carriage 104 is configured to move relative to the guide bracket member 102 via a rack and pinion assembly, which provides movement of the needle carriage 104 between a fully retracted configuration (in which the needle probes 120 are fully withdrawn and do not extend past the operating distal end of the transducer probe) and a fully deployed configuration (in which the needle probes 120 are fully extended past the operating distal end of the transducer probe). For example, the rack and pinion assembly includes a set of gears (i.e., pinion gears of the like) for receiving input from a user-operated input, which may be in the form of a knob, and converting the input (i.e., rotation of the knob) to linear motion of a rack member provided on the needle carriage 104.

As shown, guide bracket member 102 may include a pair of knobs 110 that each include a shaft collar 112, for example, which is then used for securing a pinion gear 114 thereto, all of which are operably coupled to a dowel pin 116. As more clearly shown in FIG. 5, the needle carriage 104 may include a rack member 122 defined thereon and configured to cooperatively engage the one or more pinion gears 114 that, when rotated via user input with the knob 110, result in linear movement of the needle carriage 104.

The electrosurgical device 100 may be configured to allow for different degrees of adjustment of the needle carriage 104, such as coarse movement/adjustment (i.e., faster and less precise linear movement of the needle probes between retracted and deployed configurations) and fine movement/adjustment (i.e., slower and more precise linear movement of the needle probes between retracted and deployed configurations). Accordingly, the needle probes can be advanced into the vas deferens with precise control and accuracy.

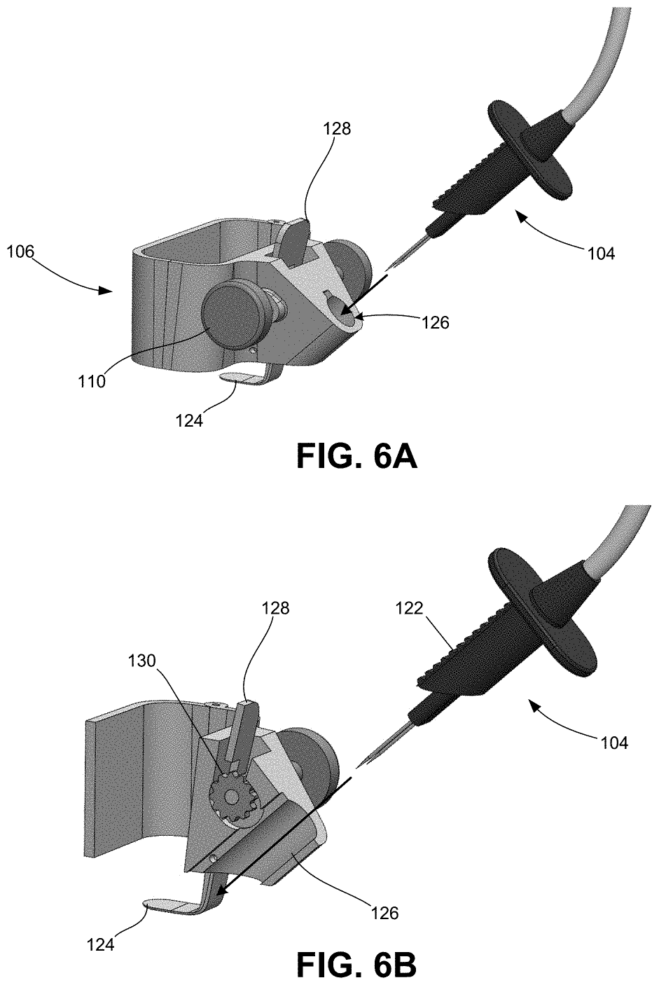

FIG. 6A is a perspective view and FIG. 6B is a perspective view, partly in section, of an exemplary embodiment of a guide bracket member and needle carriage illustrating the manner in which the needle carriage is releasably and movably coupled to the guide bracket member. As shown, the needle carriage 104 is configured to pass through aperture 126 on the guide bracket member body 106 and transition between retracted and deployed positions based on linear movement thereof. The guide bracket member 102 may further include a drive lever 128 associated with the rack and pinion assembly. The drive lever 128 is generally movable between a disengaged and an engaged state.

For example, the drive lever 128 may be operably coupled with a pinion gear 130, and, when moved to a disengaged state, the pinion gear 130 and rack member 122 are disengaged and the user can simply push or pull the needle carriage 104 to the desired position in a coarse movement/adjustment. When the drive lever 128 is moved to an engaged state, the pinion gear 130 and rack member 122 are engaged such that a user can rotate the knob 110 (associated with the pinion gear 130) to cause the needle carriage 104, and specifically the needle probes 120, to move to the desired position in a fine movement/adjustment.

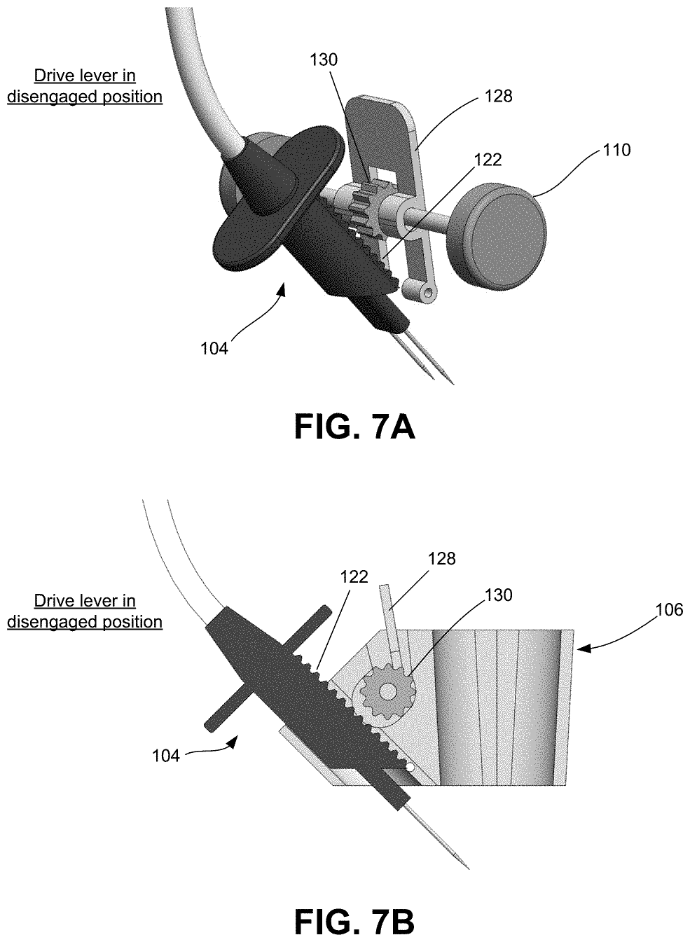

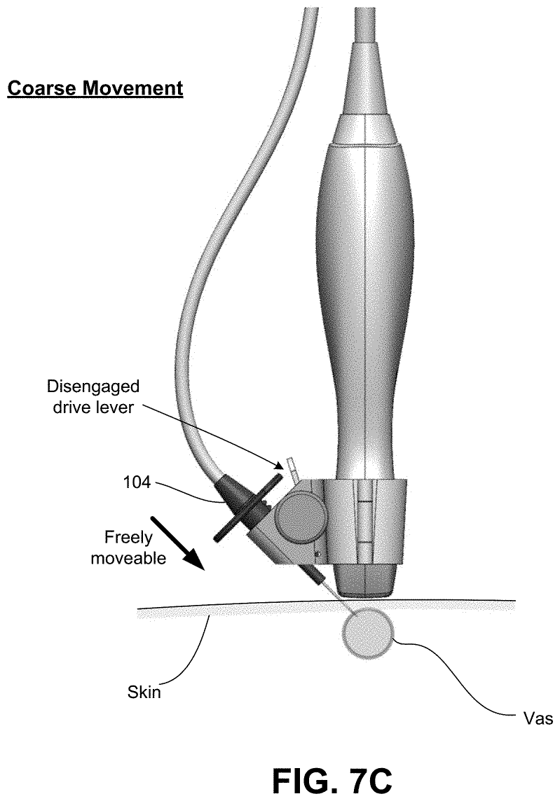

FIGS. 7A and 7B are perspective and side sectional views of portions of the guide bracket member and the needle carriage illustrating a drive lever in a disengaged position. FIG. 7C shows an ultrasound transducer probe equipped with an exemplary electrosurgical device consistent with the present disclosure and having a drive lever in the disengaged position to thereby allow for course movement/adjustment of the needle carriage, including the pair of needle probes, relative to the target site.

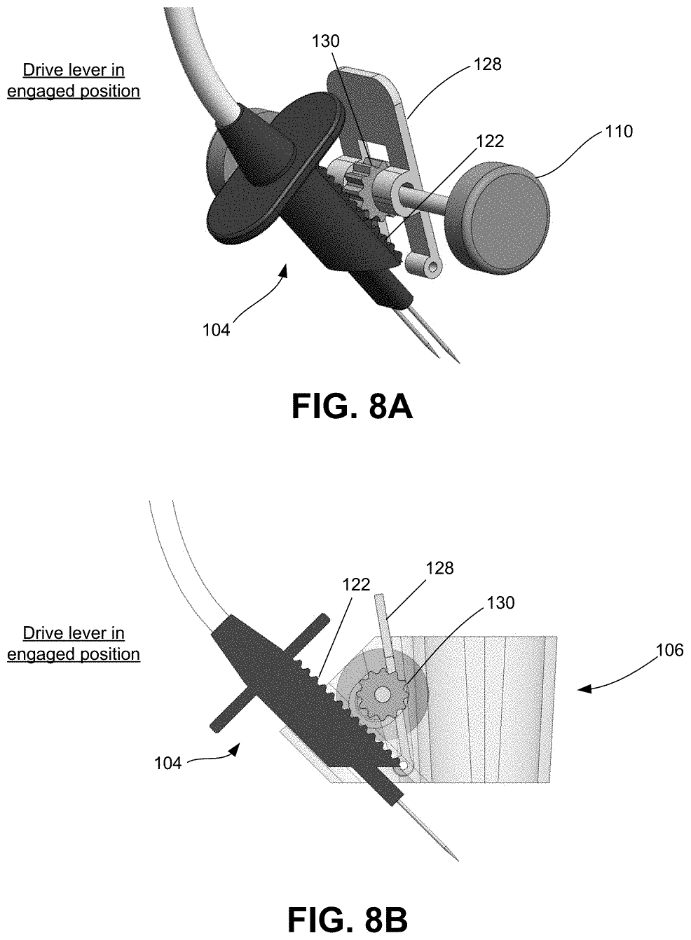

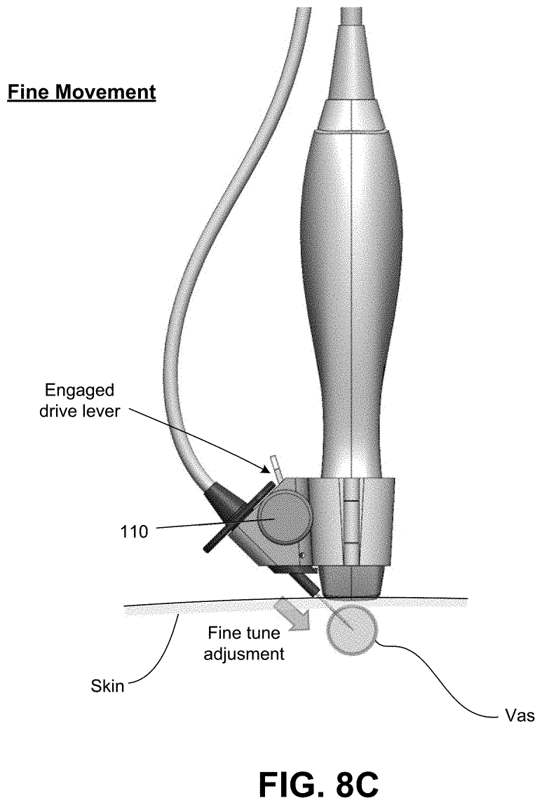

FIGS. 8A and 8B are perspective and side sectional views of portions of the guide bracket member and the needle carriage illustrating a drive lever in a engaged position.

FIG. 8C shows an ultrasound transducer probe equipped with an exemplary electrosurgical device consistent with the present disclosure and having a drive lever in the engaged position to thereby allow for fine tune movement/adjustment of the needle carriage, including the pair of needle probes, relative to the target site.

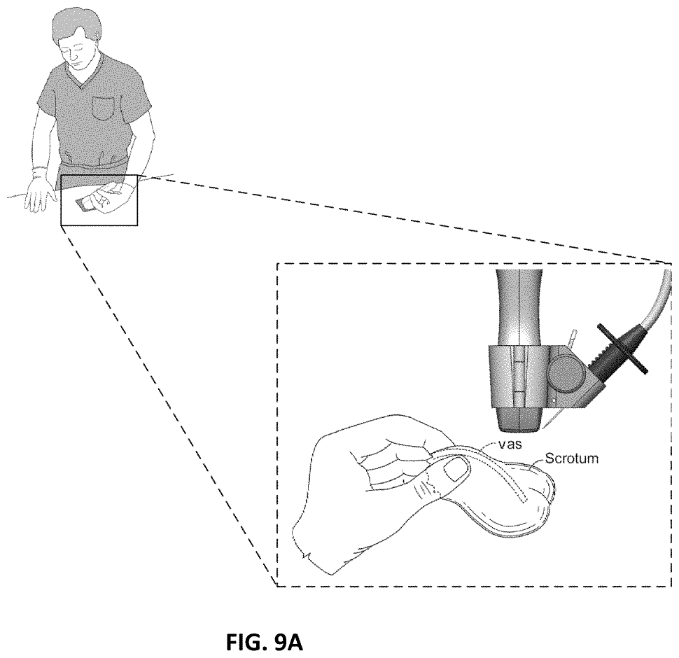

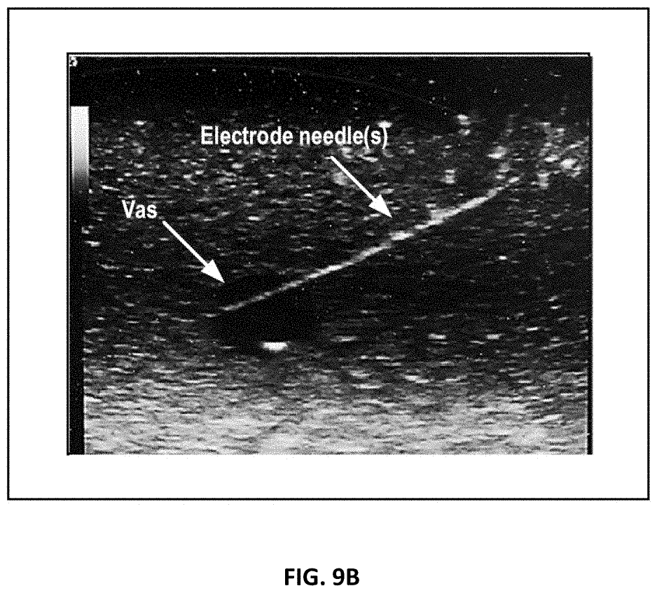

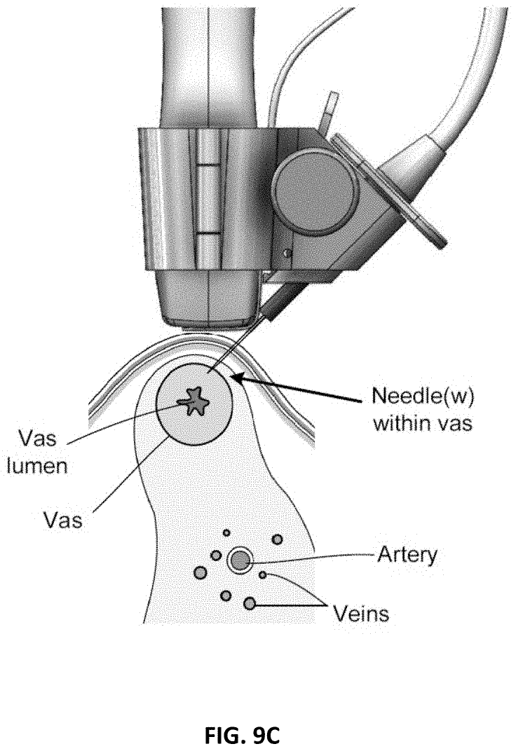

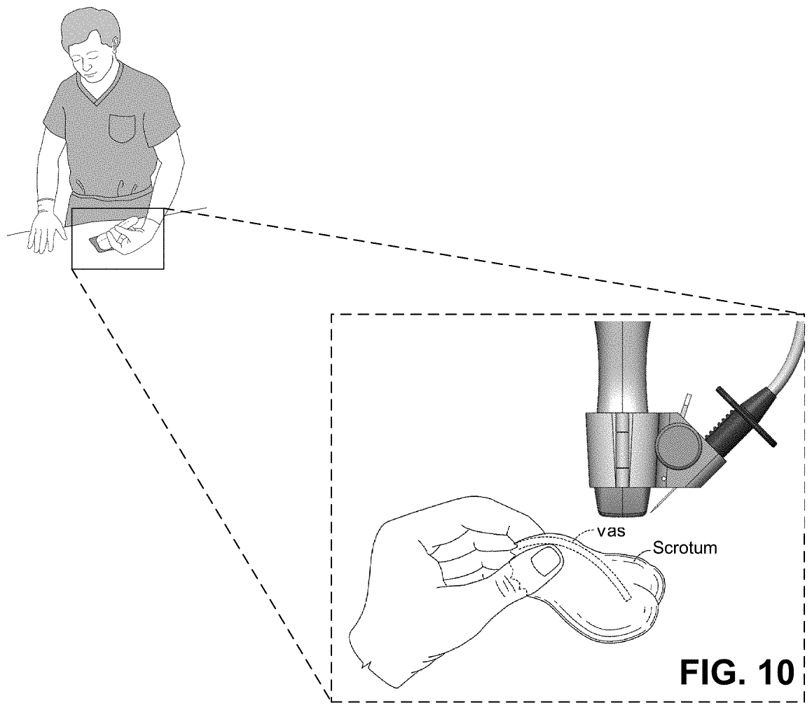

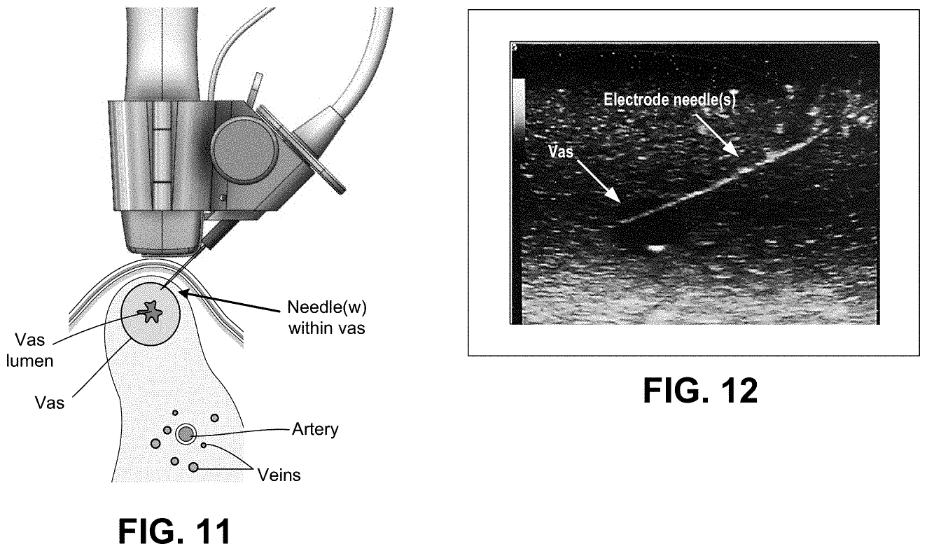

FIGS. 9A-9C are illustrated representations of steps for performing vasectomy procedure with systems consistent with the present disclosure. FIG. 9A illustrates initial grasping of the vas deferens by the physician and brought up to a position just below the surface of scrotum, in which physician may be standing on one side of the patient while the patient is lying on an exam table, and the electrosurgical device fitted over an ultrasound transducer probe is positioned relative to the vas deferens.

FIG. 9B illustrates locating and penetrating the vas deferens with the needle probes under ultrasound image guidance, in which a physician can view, in real-time, an ultrasound image (shown in FIG. 9C) of the vas deferens and advancement of the needle probes into the vas lumen. For example, the electrosurgical device, specifically the pair of needle probes of the needle carriage, are electrically coupled to an electrosurgical generator for providing bipolar low power (i.e., between 1 and 20 watts or more) RF energy delivery. The controller is communicatively coupled to the electrosurgical device 12 and the electrosurgical generator. The controller may be configured to initiate, terminate, and/or adjust delivery of RF energy from the pair of needle probes provided by the electrosurgical device.

In some embodiments, the controller may include a temperature control module (TCM), for example, that is configured to receive temperature readings from a temperature sensor (see temperature sensor 124 of FIGS. 6A and 6A) provided on the electrosurgical device and positioned adjacent to target site. In particular, the temperature control module may be configured to continuously monitor the temperature of a patient's skin at or near the target site during RF energy delivery to the vas deferens from the pair of needle probes. Upon reaching a desired maximum temperature (i.e., a predetermined temperature at which there is a high correlation with successful coagulation of the vas deferens and prior to unintended collateral damage to surrounding tissue), the temperature control module is configured to automatically terminate delivery of RF energy so as to prevent unnecessary collateral damage to surrounding tissue. For example, the TCM may be configured to automatically shut off delivery of RF energy once the temperature readings reach 50° C. or greater.

As shown, the guide bracket member is configured to receive and releasably retain the needle carriage thereto at an acute angle relative to the ultrasound transducer probe distal end. Such an orientation allows for the needle probes, particularly the penetrating distal ends thereof, to move in a direction aimed toward a target site generally centered with the distal operating end of the ultrasound transducer probe. Accordingly, when performing the procedure, the user can better target a center of the vas deferens when moving the needle probes to a fully deployed configuration, such that the tips of the probes can extend into the vas deferens and be positioned within the vas lumen for subsequent delivery of RF energy thereto.

Furthermore, the electrosurgical device may be single use, while the generator and controller are reusable. As such, the system may include a means for authenticating a given electrosurgical device to determine whether the electrosurgical device is suitable and/or authorized to operate with the controller and/or generator. In particular, the temperature control module may include an RFID reader, for example, for reading data embedded in an RFID tag associated with the electrosurgical device upon attachment of the electrosurgical device to the controller. The data from the RFID tag may be analyzed by the controller and a determination can then be made as to whether the electrosurgical device is authentic and whether it has been previously used. In the event that the electrosurgical device is determined to be authentic and not previously used, the control system allows for transmission of power to the electrosurgical device and thus a procedure can be performed using the needle probes. In the event that the electrosurgical device is determined to not be authentic or that it already has been used, the controller prevents transmission of energy to the electrosurgical device.

Accordingly, the electrosurgical device described herein is used to perform a vasectomy under ultrasound image guidance in a highly accurate and minimally invasive manner. In particular, because the electrosurgical device is directly coupled to an ultrasound transducer probe, a surgeon can perform a vasectomy in conjunction with an ultrasound imaging procedure of the target site, which allows for the surgeon to view the target site (i.e., the spermatic cord) in real-time without having to create unnecessary incisions or punctures and further locate and target the vas deferens when piercing the skin and advancing the needle probes directly into the vas lumen in a precise manner (via the fine tune advancement feature). Furthermore, the present invention provides for an automatic control over the delivery of RF energy based on continuous temperature readings at the target site to ensure that the risk of unnecessary collateral damage is minimized and that the procedure is carried out to completion. As such, the systems and methods of the present invention greatly reduce the chance of infection, bleeding, and scrotal pain associated with current techniques and devices, which may lead to greater acceptance of vasectomy by men, reducing the morbidity, mortality, and cost associated with tubal ligation.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An electrosurgical device for use in a vasectomy procedure, the device comprising:
a guide member configured to be releasably fitted to a portion of an ultrasound imaging transducer probe; and
a carriage member movably coupled to the guide member and comprising at least one pair of probes configured to deliver therapeutic energy to tissue at a target site, the pair of probes extend from a distal end of the carriage member and are movable between a fully retracted position in which a distal-most end of each probe does not extend past an operating distal end of the ultrasound imaging transducer probe and a fully deployed position in which the distal-most end of each probe extend past the operating distal end of the ultrasound imaging transducer probe and into tissue at the target site.

2. The electrosurgical device of claim 1, wherein the pair of probes are configured to deliver radiofrequency (RF) energy supplied by an electrosurgical generator.

3. The electrosurgical device of claim 2, wherein the RF energy is bipolar low power RF energy.

4. The electrosurgical device of claim 3, wherein the RF energy is between 1 watt and 20 watts.

5. The electrosurgical device of claim 4, wherein the RF energy delivered is approximately 6 watts.

6. The electrosurgical device of claim 1, wherein the pair of probes are needle probes configured to penetrate tissue.

7. The electrosurgical device of claim 1, wherein the pair of probes are spaced apart by between between 1 mm and 10 mm.

8. The electrosurgical device of claim 7, wherein the pair of probes are spaced 5 mm apart from one another.

9. The electrosurgical device of claim 1, wherein each probe has a thickness of approximately 25 gauge.

10. The electrosurgical device of claim 1, wherein the bracket member comprises one or more user-controlled inputs for controlling movement of the carriage member relative to the bracket member.

11. The electrosurgical device of claim 10, wherein the bracket member and carriage member are movably coupled to one another via a rack and pinion assembly.

12. The electrosurgical device of claim 11, wherein the bracket member comprises one or more pinion gears configured to cooperatively engage with one or more rack members defined on the carriage member, respectively.

13. The electrosurgical device of claim 12, wherein the bracket member comprises at least one knob operably associated with the one or more pinion gears such that, rotation of the at least one knob causes rotation of the one or more pinion gears and subsequent linear movement of the rack member and carriage member.

14. The electrosurgical device of claim 13, wherein the pair of probes are configured to correspondingly move in response to linear movement of the carriage member.

15. The electrosurgical device of claim 14, wherein the bracket member comprises a drive lever operably coupled with the one or more pinion gears and configured to move, upon user interaction therewith, between an engaged state and a disengaged state.

16. The electrosurgical device of claim 15, wherein, when the drive lever is in the engaged state, the one or more pinion gears and the one or more rack members are engaged with one another allowing for fine movement of the pair of probes between fully retracted and fully deployed positions based on user interaction with the at least one knob.

17. The electrosurgical device of claim 15, wherein, when the drive lever is in the disengaged state, the one or more pinion gears and the one or more rack members are disengaged from one another allowing for coarse movement of the pair of probes between fully retracted and fully deployed positions based on direct user interaction with the carriage member.

18. The electrosurgical device of claim 1, wherein the pair of probes are configured to be communicatively coupled to an electrosurgical generator via a controller configured to initiate, terminate, and/or adjust delivery of RF energy from the electrosurgical generator to the pair of probes.

19. The electrosurgical device of claim 18, wherein the controller comprises a temperature control module configured receive temperature readings from a temperature sensor provided on the electrosurgical device and positioned adjacent to the target site.

20. The electrosurgical device of claim 19, wherein the temperature control module is configured to continuously monitor temperature readings associated with skin of a patient undergoing treatment via the electrosurgical device and automatically terminate delivery of RF energy upon receiving temperature measurements reaching a maximum temperature.
